(19) United States
(12) Patent Application Publication
(10) Pub. No.: US 2003/0103966 A1
(43) Pub. Date: Jun. 5, 2003

(54) Method to induce the Th1 immune response

(76) Inventors: Johan Grooten, Lovendegem (BE); Sarita Sehra, Indianapolis, IN (US)

Correspondence Address: TRASK BRITT, P.O. BOX 2550, SALT LAKE CITY, UT, 84110, US

(21) Appl. No.: 10/310,304

(22) Filed: Dec. 5, 2002

Related U.S. Application Data
(63) Continuation of application No. PCT/EP01/06361, filed on Jun. 6, 2001.

(30) Foreign Application Priority Data
Jul. 6, 2000 (EP) 00202016.2

(51) Int. Cl. A61K 39/395

(52) U.S. Cl. 424/130.1

(57) ABSTRACT

The present invention discloses a method to induce the CD4⁺ Th1 immune response, combined with a repression of the Th2 mediated activities. The method comprises administering an IgG isotype antibody, which is not an IgG1 isotype antibody. The antibody is an IgG2a and/or IgG2b isotype anti-allergen antibody. The shift from a Th2 response towards a mixed Th1/Th2 response is useful in the treatment of diseases, such as asthma. Since the disclosed method corrects the immuno-pathological cause of the disease, the polarized Th2 response against allergen, a sustained cure from asthma may be achieved.

### CROSS-REFERENCE TO RELATED APPLICATIONS

This application is a continuation of PCT/EP01/06361, filed Jun. 6, 2001, designating the United States of America, corresponding to PCT International Patent Publication WO 01/94419 (published Dec. 13, 2001, in English), the contents of which are incorporated herein by this reference in its entirety.

### TECHNICAL FIELD

The present invention relates to a method of inducing the CD4⁺ Th1 immune response comprising administering an IgG isotype antibody, which is not an IgG1 isotype antibody. The method may be combined with repression of the Th2 mediated activities. The present invention also relates to a method to reduce eosinophilic inflammation of the airways comprising administering the antibody to a subject.

### BACKGROUND

Upon T-Cell Receptor (TCR)-ligation, Th0 cells differentiate into distinct cell subsets which are characterized by their functions and cytokine production profiles (Mosmann and Coffman, 1989). Thus, Th1 lymphocytes, characterized by the production of IL-2, IFN-γ and TNF-β, contribute to cellular immunity whereas Th2 lymphocytes, mainly involved in humoral immunity, produce IL-4, IL-5 and IL-10. Numerous examples of the consequences on disease outcomes of skewed Th1 to Th2 ratios have been reported. Further, polarized Th2 responses have been implicated in pathological situations, such as Leishmania major (Heinzel et al., 1991; Nabors et al., 1995), TBC (de Jong et al., 1997), human leprosy (Yamamura et al., 1991), and mycotic infections (Murphy et al., 1994).

The contribution of Th1 cells relative to Th2 cells to the developing autoimmune response determines whether or not the autoimmune response leads to clinical disease (Racke et al., 994; Racke et al., 1995; Leonard et al., 1995). Chronic autoimmune graft-versus-host disease, which develops after the administration of mismatched lymphoid cells, can be prevented by switching a Th2 response to a Th1 response through the administration of IFN-γ at the time of cellular transfer (Donckier et al., 1994). Roussel et al. (1996) describe that the inefficiency of the immune response against a human glioma is caused by the presence of activated tumor-infiltrating lymphocytes, characterized by a predominant type 2 lymphokine production. These cytokines do not promote a tumoricidal immune response and, therefore, do not counteract the growth of the tumor.

In allergic asthma, a predominant Th2 response has been noted (Vogel, 1997). Asthma is characterized by a heterogeneous collection of clinical symptoms including reversible airway narrowing, airway hyperreactivity, and eosinophilic inflammation of the airways. Due to the chronic nature of asthma, structural and functional changes in the organ will occur in the long-term and may result in airway remodelling and further amplification of the syndrome. Further, sensitization to airborne environmental allergens leads to atopy and is a major risk factor for asthma (reviewed in Holt et al., 1999).

In sensitized individuals, exposure to the aeroallergen will trigger an acute response within minutes, resulting in airway constriction and difficult breathing. Interaction of the allergen with allergen-specific IgE antibodies bound to various effector cells through the Fcε receptor I provides the trigger for this acute reaction. The secreted inflammatory mediators recruit eosinophils, mast cells, T lymphocytes and other circulating leukocytes to the site(s) of allergen challenge. Besides causing a recurrence of symptoms, the cellular infiltration of effector cells will persist upon chronic exposure to allergen and lead to chronic eosinophilic inflammation of the airways which is characteristic for asthma (reviewed in Wills-Karp, 1999 and Galli, 2000).

Specific cytokines, various inflammatory mediators and allergen-specific IgE antibodies contribute to the complex pathogenesis of asthma. However, increasing evidence indicates that Th2 cell-derived cytokines are important in the generation and persistence of the disorder. Thus, IL-4 and IL-13 are important in causing B lymphocytes to produce allergen-specific IgE. IL-3 controls the induction of mast cell proliferation and the recruitment of lymphocytes, mast cells and basophils. IL-5 is involved in the growth and differentiation of eosinophils and B lymphocytes, while IL-9 promotes the growth and differentiation of mast cells. IL-10 inhibits IFN-γ production and classical activation of macrophages (reviewed in Corry and Kheradmand, 1999).

Accordingly, Th2-derived cytokines, along with IgE-mediated activities, represent important therapeutic targets. Strategies aimed at eliminating or neutralizing these activities are being actively pursued. These strategies involve the administration of neutralizing or antagonistic anti-cytokine or anti-IgE antibodies, administration of soluble cytokine receptors or peptido-mimetics of cytokine receptors. For instance, PCT International Patent Publication WO 90/04979 describes a method of preventing or reducing eosinophilia comprising administering an antagonist to human IL-5, such as a monoclonal antibody against IL-5. However, the disclosed methods are not antigen specific. As a result, the disclosed methods will affect the targeted allergic immune response as well as immune responses against unrelated antigens.

Alternative strategies for treating allergic diseases include selective suppression of the anti-allergen immune response. These strategies are based on some form of active vaccination using injections of crude or purified allergen preparations which result in hyposensitization. Classically, the subcutaneous route of administration is used for this type of immunotherapy.

A more recent approach for immunotherapy, the so-called “Saint-Remy technique” (European Patent Office Publications EP 0178085 and EP 0287361), uses autologous IgG antibodies complexed in vitro to the relevant allergen(s). This approach generates fewer side effects since smaller amounts of allergen are applied. Hyposensitization has proven to be somewhat effective in treating allergic diseases including allergic rhinitis and asthma. However, numerous difficulties exist with this form of treatment. For instance, treatment schedules are cumbersome and prolonged courses of treatment are necessary, resulting in low patient compliance. Further, since the precise immune mechanism is not known, the cause of therapeutic failure usually cannot be established.

Various improvements on the vaccination approach have been described in an effort to render hyposensitization more effective. These improvements include encapsulation in or covalent attachment of the allergen to liposomes (U.S. Pat. No. 5,049,390), covalent attachment of the allergen to a saccharide (U.S. Pat. No. 5,073,628), application of adjuvants that suppress formation of IgE antibodies and promote formation of IgM and IgG antibodies and others. In one example, the adjuvant includes a, glycolipid extracted from maize tissue (U.S. Pat. No. 4,871,540) along with preparations containing live Mycobacterium bovis, Bacillus Calmette-Guerin or mycobacterial cell wall products (Azuma et al., 1976; Yang et al., 2000). These methods, as well as methods wherein the allergen is first modified by coupling to various bridging molecules (such as antibodies) and subsequently administered to the recipient, are described in, for instance, PCT International Patent Publication WO 97/07218 and have the drawback that the methods encompass administration of an allergen-containing composition to individuals that exhibit various degrees of atopy and/or anaphylaxis. Therefore, the individuals are at risk of developing immediate hyperresponsiveness and/or anaphylactic shock in response to the treatment.

Yamauchi et al. (1983) describe that intravenous administration of specific IgG2 antibody prior to challenge with antigen inhibited the IgE induced bronchial response in a model of allergic asthma. The authors suggested that a direct competition between IgG2 and IgE for the antigen is responsible for the inhibition of the IgE induced bronchial response by blocking the trigger required for the acute reaction. This treatment would, therefore, be suitable for a symptomatic treatment, but not a cure for the allergic asthma.

### SUMMARY OF THE INVENTION

An approach for the treatment of allergic diseases based on the conversion of the anti-allergen pathogenic response to a benign and persistent immune response is disclosed. An anti-allergen antibody of the IgG isotype, which is substantially free of allergen and not an IgG 1 isotype, was found to induce the Th1 response and repress the Th2 related activities.

The disclosed findings imply a conversion of the polarized Th2 cell response, characteristic for allergic asthma, to a mixed Th1/Th2 response or a predominant Th1 response. Due to the pivotal role of Th2-cell derived cytokines in allergic asthma, the conversion reduces allergic asthma due to the diminished production of the causative Th2 cytokines and the mutual antagonistic activity of Th1 and Th2 cytokines. Since this approach is antigen specific, it does not abrogate ongoing beneficial Th2 responses against unrelated antigens. The approach also has promise as a substantial cure from asthma in addition to being a symptomatic treatment since the fundamental cause of the disease is eliminated, namely the anti-allergen Th2-polarized immune response. Further, since the treatment does not require administration of allergen, in a native form or modified, the disadvantages and health risks intrinsic to active vaccination strategies are avoided.

In a first aspect of the invention, a method to induce the CD4⁺ Th1 immune response comprising administering a compound that binds allergen and directs the allergen to an antigen-presenting cell (APC) to induce and/or support a Th1 response and counteract a Th2 response is disclosed. The compound may be administered intranasally. The compound allows spontaneously inhaled environmental allergen to be directed to the antigen-presenting cells that induce and/or support Th1 responses and counteract a Th2 response. Thus, the pathological Th2 response is converted into a beneficial mixed Th1/Th2 response or a predominant Th1 response without the requirement for enforced exposure of the asthmatic individual to an increased allergen load. In this manner, the risk of treatment-induced anaphylaxis is abolished while an antigen-specific and sustained suppression of asthma is generated. Moreover, the shift to the mixed Th1/Th2 response or predominant Th1 response is persistent in time and allows the allergic reaction to be cured, rather than merely being a symptomatic treatment.

Different types of APCs may steer differentiation of the CD4⁺ T cells into Th1, Th2, or Th1 and Th2 effectors. Dendritic cells induce the development of Th1 or Th2 cells depending on the state of differentiation and/or the presence of factors such as IFN-γ or prostaglandin E2 in the microenvironment of the Th1 or Th2 cells (Macatonia et al., 1995; Ronchese et al., 1994; Kalinski et al., 1999). In contrast, B cells seem to support the induction and expansion of Th2 cells (Gajewski et al., 1991).

The involvement of macrophages in initiating cognate immunity has long remained elusive. Although macrophages are dedicated APCs in vitro, the macrophages exert this activity only after treatment with IFN-γ and appear to be mainly involved in non-specific inflammatory responses. However, macrophages are an important source of IL-12 and may favor the development of Th1 cells which is supported by the observation that macrophage depletion in mice shifts an expected Th1 response to a Th2 response (Brewer et al., 1994). Moreover, PCT International Patent Publication WO 99/21968 describes that ex vivo loaded antigen-presenting macrophages may be used to influence the CD4⁺ Th1/CD4⁺ Th2 balance towards Th1 reactivity.

In one embodiment, a method to induce the CD4⁺ Th1 immune response comprising administering a compound that can bind an allergen and direct the allergen to an antigen-presenting cell to induce and/or support a Th1 response and counteract a Th2 response is disclosed. In this embodiment, the antigen-presenting cell is a macrophage, such as an IFN-γ activated macrophage.

In another embodiment, a method to induce the CD4⁺ Th1 immune response comprising administering a compound that can bind an allergen and direct the allergen to an antigen-presenting cell to induce and/or support a Th1 response and counteract a Th2 response is disclosed. In this embodiment, the compound is an IgG isotype antibody that is substantially free of allergen and is not an IgG1 isotype antibody. The antibody may be an anti-allergen antibody.

In yet another embodiment, a method to induce the CD4⁺ Th1 immune response comprising administering a compound that can bind an allergen and direct the allergen to an antigen-presenting cell to induce and/or support a Th1 response and counteract a Th2 response is disclosed wherein the compound is an IgG2 isotype antibody.

In still another embodiment, a method to induce the CD4⁺ Th1 immune response comprising administering a compound that can bind an allergen and direct the allergen to an antigen-presenting cell to induce and/or support a Th1 response and counteract a Th2 response is disclosed wherein the compound is a bispecific or multispecific antibody. In this embodiment, at least one specificity of the antibody is directed against the allergen and at least another specificity of the antibody is directed against the antigen-presenting cell, wherein the antigen-presenting cell is a macrophage or an IFN-γ activated macrophage. The antigen may be directed against the low affinity receptor Fcγ receptor II or against CD14 on the macrophage. Methods to produce bispecific or multispecific antibodies are known by the person of ordinary skill in the art and have been described, for example, in PCT International Patent Publication WO 99/37791 and by Merchant et al. (1998)

In another aspect of the invention, a method to reduce aeroallergen-induced inflammatory responses in the airways comprising administering a compound that can bind allergen and direct the allergen to an antigen-presenting cell to induce and/or support a Th1 response and counteract a Th2 response is disclosed. In this aspect, the reduction of aeroallergen-induced inflammatory responses is persistent. The compound may be administered intranasally and the antigen-presenting cell may be a macrophage. The compound may be an IgG isotype antibody substantially free of allergen, wherein the IgG isotype antibody is not an IgG1 isotype antibody. The IgG isotype antibody may be an anti-allergen antibody.

In one embodiment of this aspect, a method to reduce aeroallergen-induced inflammatory responses in the airways comprising administering a compound that can bind an allergen and direct the allergen to an antigen-presenting cell to induce and/or support a Th1 response and counteract a Th2 response is disclosed, wherein the compound is an IgG2 isotype antibody.

In yet another aspect of the invention, a pharmaceutical composition for treating a disease characterized in that the natural CD4⁺ Th1/CD4⁺ Th2 balance is biased towards a Th2 response and/or which can be treated by shifting the balance towards a Th1 response is disclosed. The pharmaceutical composition comprises one or more IgG isotype antibodies which are substantially free from other isotype antibodies and substantially free from allergen. In the disclosed pharmaceutical composition, the IgG isotype antibody is not an IgG1 isotype antibody. Diseases of the disclosed type are known by the person skilled in the art and include, but are not limited to, allergic asthma, allergic rhinitis, airway hyperreactivity and eosinophilic airway inflammation. In this aspect, the antibody is an anti-allergen antibody and/or the pharmaceutical composition is configured for intranasal administration.

In still another aspect of the invention, an IgG isotype antibody is used to manufacture a medicament. The medicament is designed to treat a disease in which the natural CD4⁺ Th1/CD4⁺ Th2 balance is biased towards a Th2 response and/or which may be treated by shifting the balance towards a Th1 response, wherein the IgG isotype antibody is not an IgG1 isotype. In this aspect, the antibody is an anti-allergen antibody and is directed against antigenic structures of causative agents of the disease. In one embodiment of this aspect, an IgG isotype produced in accordance with the principles of the invention is used to treat a disease such as allergic asthma, allergic rhinitis, airway hyperreactivity or eosinophilic airway inflammation.

### BRIEF DESCRIPTION OF THE DRAWINGS

FIG. 1. Increment of Ovalbumin (OVA)-specific IgE titers by aerosol challenge of BALB/c mice, sensitized by repetitive OVA injections.

FIG. 2. Bronchial Alveolar Lavage (BAL) cellular content from mice sensitized and challenged with OVA (OVA/OVA). Controls for induction of hyperresponsiveness are placebo treated mice (PBS/PBS) and mice that were sensitized, but not challenged, with OVA (OVA/PBS). Mean values of the respective experimental groups are shown (n=3).

FIG. 3. Recovery of anti-hCat IgG from lungs after administration by aerosol or intranasal instillation.

FIG. 4. Clearing from lungs of anti-OVA IgG, instilled by intranasal route.

FIG. 5. Detection of cell-bound anti-OVA IgG in BAL cells of C57BL/6 mice. Fluorescent-labelled antibody was administered by intranasal route and the presence of cell-bound Ig was analyzed by flow cytometry (line). Filled histograms represent autofluorescence of non-labelled cells.

FIG. 6. OVA-sensitized mice were treated twice with the indicated amounts of anti-OVA IgG administered by intranasal route. Time of treatment was 2 h before the first and the fifth aerosol. The number of BAL eosinophils is expressed as % relative to control mice that received PBS. Bars represent individual mice.

FIG. 7. OVA-sensitized mice received by intranasal or intravenous route a single administration of 100 μg anti-OVA IgG by intranasal or intravenous route, 2 h before the first aerosol exposure. The number of BAL eosinophils is expressed as % relative to control mice that received PBS. Bars represent individual mice.

FIG. 8. Total cells and eosinophils in BAL of mice after single OVA-alum sensitization (experiment 1) or double OVA-alum sensitization (experiment 2). The amount of anti-OVA IgG used is indicated in the figure.

FIG. 9. Anti-OVA IgG titer in serum after a second round of aerosol challenge. Mice were triple sensitized with OVA-alum, treated twice with 50 μg IgG, with a first round of antigen aerosol challenge 2 hours after each treatment and a second round 6 days after the IgG treatment. The experimental outline is shown in FIG. 11.

FIG. 10. Evidence for persistence of the anti-inflammatory effect upon aerosol challenge after stopping the treatment: short term protection. Total cells and eosinophils in BAL of mice after triple OVA-alum sensitization and intranasal treatment with 2×50 μg IgG, followed by aerosol antigen exposure 2 hrs after each IgG treatment. BAL is harvested 2 days after the last IgG treatment. In the drawing, OVA-alum represents OVA-alum sensitization, Ab represents IgG treatment, aerosol represents antigen challenge, and BAL represents BAL harvest.

FIG. 11. Evidence for persistence of the anti-inflammatory effect upon aerosol challenge after stopping the treatment: persistent protection. Total cells and eosinophils in BAL of mice after triple OVA-alum sensitization and intranasal treatment with 2×50 μg IgG, followed by aerosol antigen exposure 2 hrs after each IgG treatment. An additional aerosol antigen exposure is given 6 days after the last IgG treatment. BAL is harvested 8 days after the last IgG treatment. Abbreviations are the same as those in FIG. 10.

### DETAILED DESCRIPTION

DEFINITIONS. The following definitions are set forth to illustrate and define the meaning and scope of various terms used to describe the invention herein.

“IgG2 isotype antibody” as used herein refers to an isotype antibody derived from a polyclonal or a monoclonal preparation. The isotype antibody may be purified to a degree such that it is free from immunological active amounts of antibodies of other isotypes or of other immunological active compounds.

“Substantially free of allergen” refers to the ratio of the number of antibodies to the number of antibody-binding epitopes which bind to the antibodies as measured in vitro before administration. The ratio may be 10/1, 100/1, or even 1,000/1.

One or more IgG isotype antibodies “substantially free of other isotype antibodies” means that the ratio of the total number of IgG isotype antibodies to the total number of non-IgG isotype antibodies, as determined in vitro, before administration is at least 10/1, but maybe 100/1 or even 1,000/1.

“Environmental allergen” as used herein refers to allergen to which an animal subject, including a human, is exposed to by external contact, such as inhalation.

“Aeroallergens” include, without limitation, pollen. The pollen may originate from gymnosperms, dicotyledonous angiosperms, monocotyledonous angiosperms, dust mite antigens and mold antigens, such as Alternaria antigens.

“A persistent reduction” of aeroallergen-induced inflammatory response is a reduction wherein, after contact with the allergen, a significant decrease in inflammatory response is noticed. The reduction may occur even after stopping the treatment for at least four days and even after stopping the treatment for at least six days. The significance of the decrease may be evaluated by comparing the treatment to a result obtained in a persistent reduction of aeroallergen-induced inflammatory response with a placebo treatment.

### EXAMPLES

### Methods Used in the Examples

Mouse strains: In all experiments, unless otherwise indicated, BALB/c mice were used.

Aeroallergen: Ovalbumin (OVA).

Induction protocol: Sensitization by repeated injection of OVA. Alternatively, mice were sensitized by injection of 10 μg OVA adsorbed with 1 mg Al(OH)₃ (OVA-Alum). Dependent on the experiment and degree of sensitization desired, mice were injected with OVA-Alum once on day 0 or received an additional injection on day 7 and day 14. Challenge was by 8 consecutive exposures to nebulized OVA over 8 days, unless otherwise indicated.

Parameters monitored: Number of BAL cells in individual animals; composition of BAL regarding numbers of eosinophils, macrophages, CD4⁺ T cells and CD8⁺ T cells; number of cytokine-positive CD4⁺ T cells from BAL following in vitro activation with anti-CD3 and anti-CD28 monoclonal antibody; cytokine concentration in the supernatant of the above described T cell cultures collected after 24 hrs; serum titers of OVA-specific IgE, IgG1, IgG2a and IgG2b antibodies (OVA-specific Elisa).

Anti-OVA antibodies: Mouse monoclonal anti-OVA antibodies of the isotypes IgE, IgG1, IgG2a and IgG2b were isolated in the laboratory. Anti-OVA IgE-containing crude hybridoma culture supernatant was used as internal standard for OVA-specific IgE Elisa. The various anti-OVA IgG monoclonal antibodies were similarly used as internal standard for specific Elisa. In addition, cultures of the corresponding hybridomas were expanded for large-scale antibody production followed by purification of the monoclonal antibody. All preparations were found to be free of endotoxin.

Route of administration of anti-OVA antibody: Antibodies were administered either by intravenous (i.v.) injection or by intranasal instillation.

### Example 1

### Murine Experimental Model for Persistent Atopic Asthma in Humans

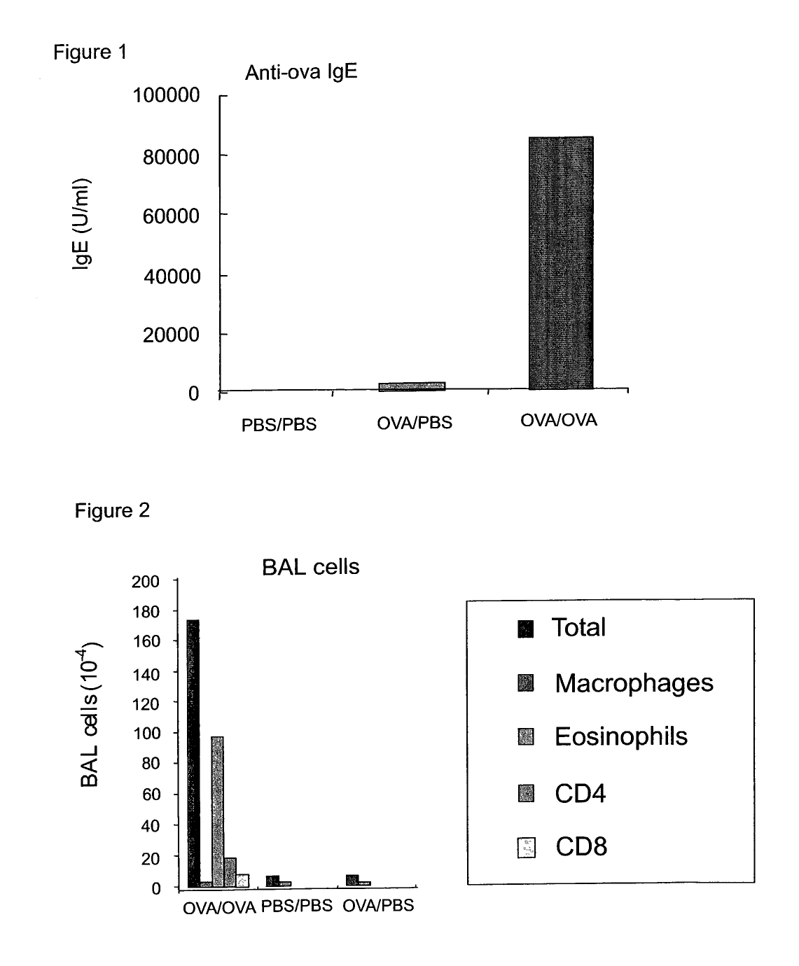

A well-established experimental model for allergic asthma includes sensitization of BALB/C mice to the protein antigen OVA, followed by challenging the sensitized mice by repeated exposure to nebulized OVA (Hofstra et al., 1998). Sensitization was achieved by 7 intraperitoneal injections of 10 μg OVA in PBS given on alternate days. Exposure of treated mice, 3 weeks after the last injection, to inhaled OVA resulted in induction of atopy which was apparent from strongly increased serum titers of anti-OVA IgE (FIG. 1). This IgE response was accompanied by a strong increment of cellular infiltration in the lungs. The cell infiltrate included mainly eosinophils, CD4⁺ and CD8⁺ T lymphocytes, and macrophages (FIG. 2). Both responses to inhaled allergen, namely induction of atopy and eosinophilic airway inflammation, are characteristic of allergen-induced asthma and as a consequence represent a valid experimental model for the human disease.

### Example 2

### Advantages of Intranasal Administration of IgG Antibody

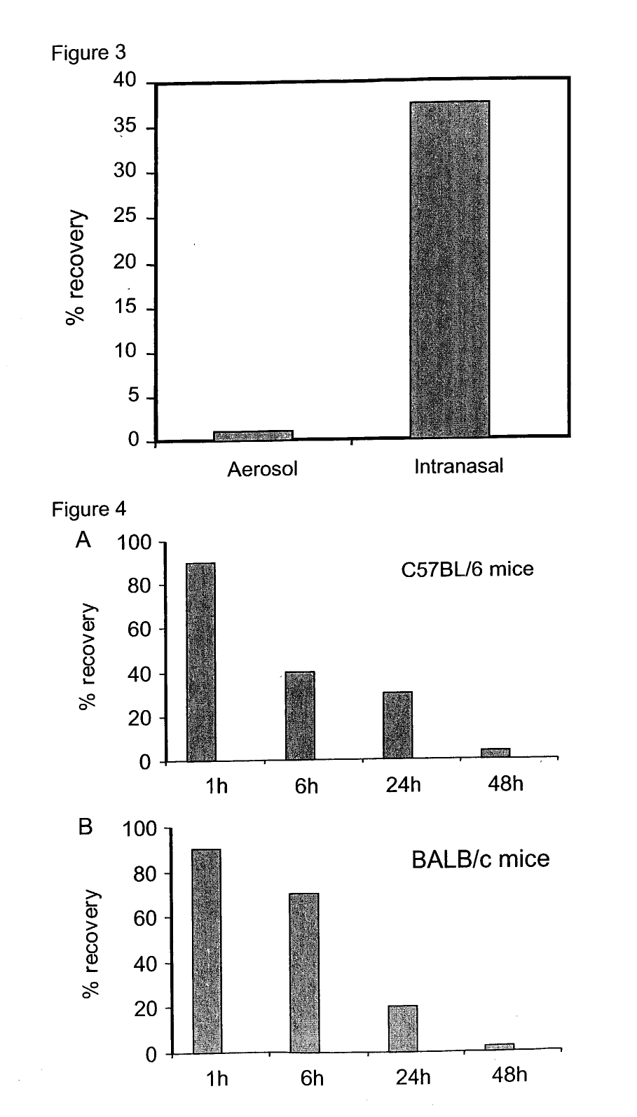

An essential feature of the approach described herein relates to the spontaneous formation of antibody-allergen immune complexes formed as inhaled allergen reaches the airways. Therefore, administration of antibody specifically to the airways is important. The feasibility of introducing antibodies to the lungs by aerosol or by intranasal instillation was investigated. The presence of functional anti-human catalase IgG antibody (anti-hCat) in the BAL was measured by specific Elisa after administration of the antibody by aerosol or intranasal instillation. As shown in FIG. 3, administration by aerosol allowed recovery of functional antibody and intranasal instillation allowed nearly 40% recovery of functional antibody. Control experiments showed the dramatic loss of functional antibody in the BAL after aerosol administration which reflected loss of function of the antibody rather than inadequate inhalation. Altering the pressure used for aerosol and/or the concentration of the antibody did not lead to significant gains in antibody stability. Accordingly, intranasal instillation was chosen as an administration method for delivery of antibody to the upper airways.

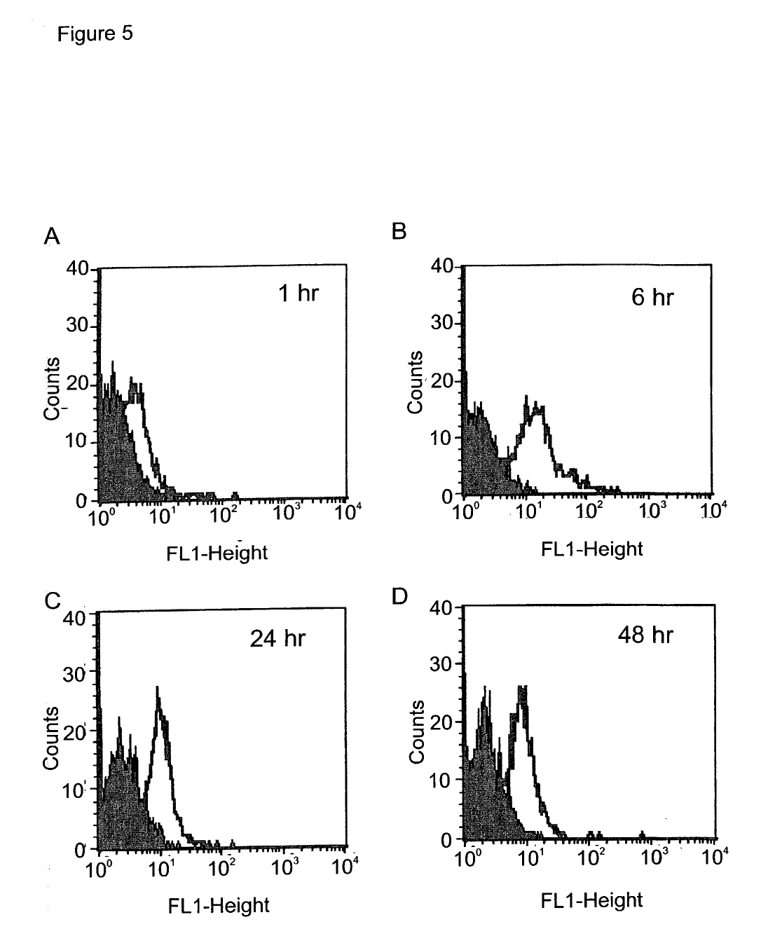

A second parameter that was established dealt with the time of retention of antibody in the lung which is used to define the time range wherein the administered antibody may exert its presumed effects. OVA-specific Elisa on BAL fluid of mice that received anti-OVA IgG by the intranasal route showed a slow clearance of free antibody while significant titers were still detectable after 24 h (FIG. 4). After 48 h, most of the antibody appeared cleared from the lungs. To determine whether cell-bound antibody exhibited a similar clearance rate, fluorescent-labelled antibody was administered and the presence of cell-bound fluorescence was measured on BAL cells using flow cytometry. In C57BL/6 mice, cell-bound antibody was detectable within 1 h after intranasal instillation and reached maximal intensity after 6 h (FIG. 5). Unlike free antibody, cell-bound antibody remained detectable 48 h after administration. A similar result was obtained with BALB/c mice. From these results, it may be concluded that intranasal administered antibody may exert its local effects in the airways within a time span of 24 h to 48 h.

### Example 3

### Reduction of Allergen-induced Airway Inflammation is IgG2 Dependent

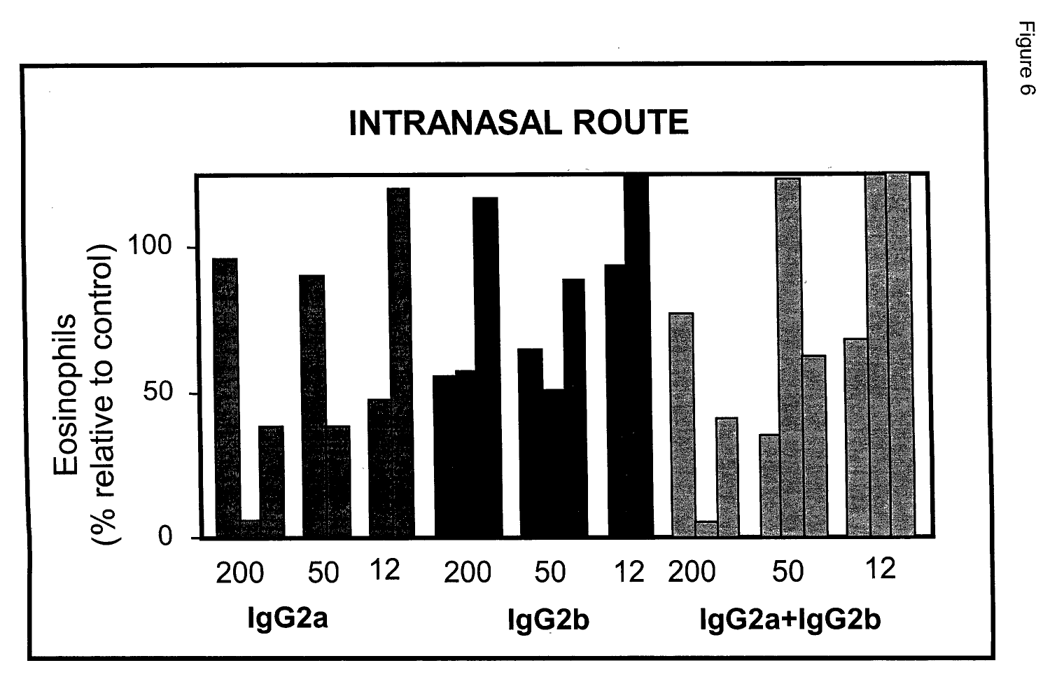

In another set of experiments, it was verified whether administration of anti-allergen IgG antibodies to sensitized mice 2 hrs before challenge with aerosol affects the airway inflammatory response. Preliminary experiments indicated that an antibody dose range of about 50 to 200 μg IgG antibody was effective (FIG. 6). The following experiments were used for an antibody dose of 100 μg and the following experimental parameters were varied:

The IgG isotype administered was IgG1, IgG2a, or IgG2b;

The number of administrations was once (2 h before the first exposure to aerosol) or twice (an additional administration of antibody 2 h before the 5^{th} aerosol exposure); and

The route of administration was intravenous or intranasal.

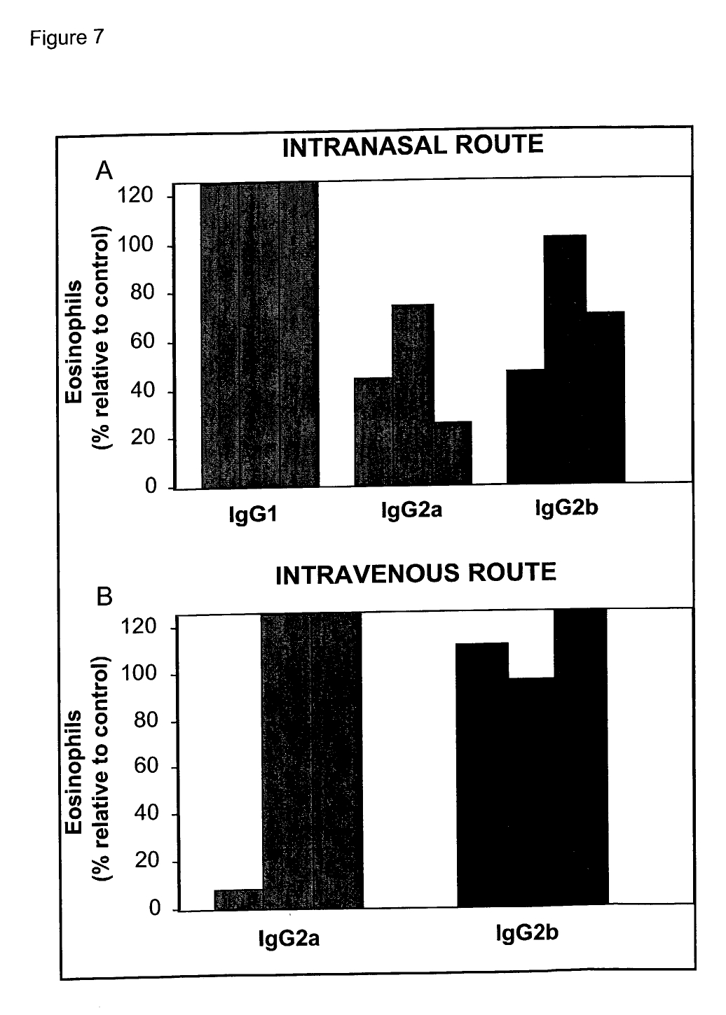

The extent of eosinophilia in the BAL was analyzed. Since eosinophilia is the major indicator of allergen-induced airway inflammation, the extent of eosinophilia revealed a pronounced reduction in the conditions where IgG2 antibodies were administered to the lungs by intranasal instillation (FIG. 7, upper panel). IgG2a appears to be a potent IgG2 isotype in generating this protective effect. In contrast, intranasal administration of IgG1 appeared to have no protective effect. Administration of the same antibodies by the intravenous route had no, or only marginal, effects on the degree of eosinophilia (FIG. 7, lower panel).

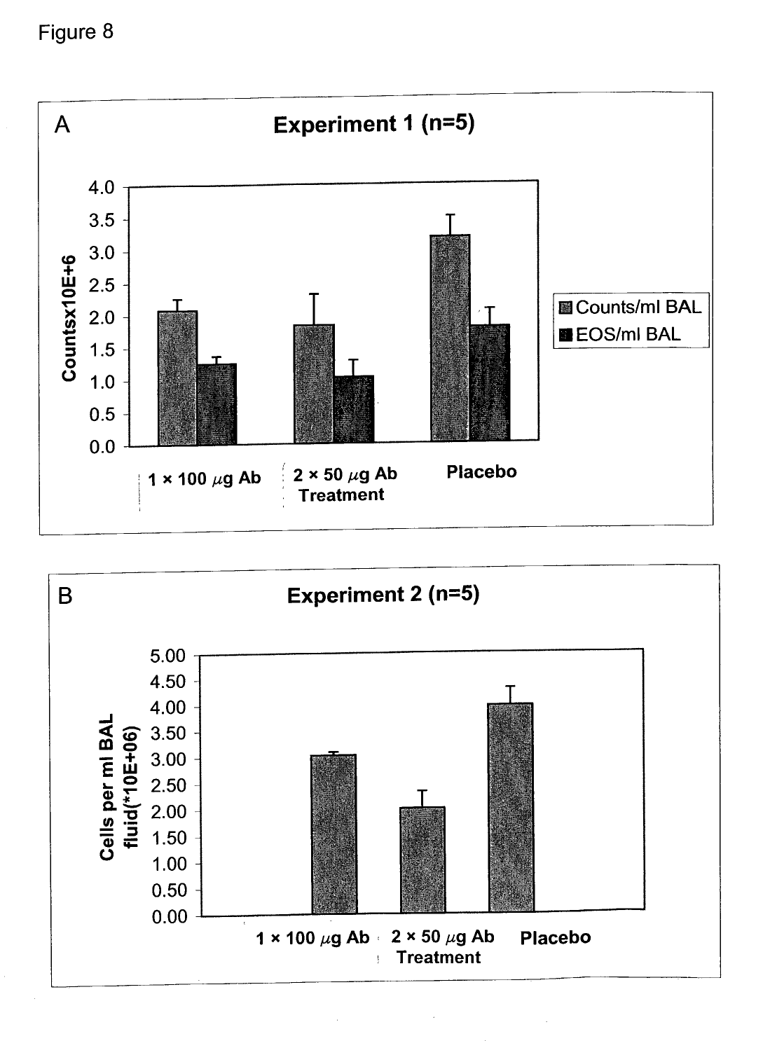

A comparison in two separate experiments between the same IgG2a antibody dose (100 μg) given in a single administration or divided over two separate administrations of 50 μg each revealed a diminished eosinophilia and diminished cell infiltration in the airways with both treatment schedules (FIG. 8). However, two separate administrations of 50 μg IgG2a each produced a more pronounced reduction in both independent experiments of allergen-induced airway inflammation as compared to a single administration of 100 μg IgG2a antibody.

### Example 4

### Analysis of the Serum Titers Induced by a First Round of Aerosol Challenge

Analysis of the serum titers of OVA-specific IgE, IgG1, IgG2a and IgG2b induced by challenge with OVA aerosol revealed no significant changes between the various experimental groups (Table I). Thus, despite the presence of OVA-specific IgG antibodies in the airways, a challenge with inhaled antigen induced a secondary antibody response similar to the antibody response induced in placebo-treated mice. This result indicates that the reduced airway inflammation observed in the IgG2-treated mice did not result from molecular avoidance or immune exclusion of the acroallergen by the administered allergen-specific antibodies as was previously reported for allergen-specific IgA (Schwarze et al., 1998).

TABLE I
Anti-OVA Ig-serum titers induced by OVA aerosol challenge of
treated and untreated sensitized mice.
ND = not determined.
i.n. = intranasal administration.
In the columns labeled IgE, IgG1, IgG2a, and IgG2b, the anti-OVA
Ig-serum titer of the indicated antibody type is presented.

| | Mouse | | | | |
|---|---|---|---|---|---|
| Treatment (i.n.) | number | IgE | IgG1 | IgG2a | IgG2b |
| 100 μg IgG1 Ab | 1 | 66667 | 1000 | ND | ND |
| 100 μg IgG1 Ab | 2 | 66667 | 1000 | ND | ND |
| 100 μg IgG1 Ab | 3 | 50000 | 1000 | ND | ND |
| No antibody | 10 | 66667 | 1000 | ND | ND |
| No antibody | 11 | 50000 | 600 | ND | ND |
| No antibody | 12 | 50000 | 700 | ND | ND |
| 100 μg IgG2a Ab | 1 | 44444 | 2778 | 1375 | 20 |
| 100 μg IgG2a Ab | 3 | 94444 | 4444 | 2500 | 71 |
| 100 μg IgG2a Ab | 4 | 44444 | 2778 | 1375 | 36 |
| No antibody | 18 | 87500 | 5600 | 3333 | 160 |
| No antibody | 19 | 75000 | 6400 | 1667 | 50 |
| No antibody | 20 | 75000 | 7200 | 3333 | 60 |
| 100 μg IgG2b Ab | 1 | 33333 | 3750 | 275 | 120 |
| 100 μg IgG2b Ab | 2 | 83333 | 10000 | 775 | 280 |
| 100 μg IgG2b Ab | 3 | 46667 | 6500 | 350 | 140 |
| No antibody | 19 | 33333 | 4000 | 225 | 119 |
| No antibody | 20 | 17667 | 2750 | 25 | 35 |
| No antibody | 21 | 28333 | 4000 | 250 | 143 |

Based on the data in Table I, an active process involving a modulation of the allergen-induced immune response by the administered IgG2 antibodies appears to be responsible for the attenuation of the airway inflammatory response to allergen. Also, the recurrent response pattern observed in example 3 with various administration schedules of allergen-specific IgG antibodies indicates an alternative modulation of the anti-allergen immune response. Thus, all treatments involving intranasal instillation of IgG2, but not IgG1, antibodies consistently resulted in a diminished airway inflammatory response to inhaled allergen whereas the intravenous route of administration did not produce this consistent response pattern. The discrepancy between administration routes indicates that the protective effect of the allergen-specific IgG2 antibodies requires interaction of the antibody with the allergen at the site of allergen entry. As the protection is not the result of shielding of the immune system from the allergen by the administered antibody, an active, not passive, mechanism must be responsible for the observed reduction in inflammation. The observations, specifically the dependence of the protective effect on intranasal instillation and its occurrence despite contact of the immune system with the allergen, indicate that the disclosed method modifies the nature of the anti-allergen immune response and is valid for obtaining a sustained cure for asthma, rather than a symptomatic treatment.

### Example 5

### Analysis of the Serum Titers Induced by a Second Round of Aerosol challenge.

The absence of decreased IgE and IgG antibody responses in the treated animals, despite a marked reduction of the inflammatory airway response, can be explained as follows. The observed antibody titers reflect the activation of antibody-producing memory B lymphocytes by allergen, wherein the B lymphocytes are generated during the preceding sensitization. Antibodies derived from newly generated antibody-producing B cells marginally contribute to the antibody response due to the short period (7 days) between the aerosol challenge and the serum collection. However, upon renewed challenge with allergen, memory B cells derived from the newly generated antibody-producing B cells will significantly contribute to the antibody response.

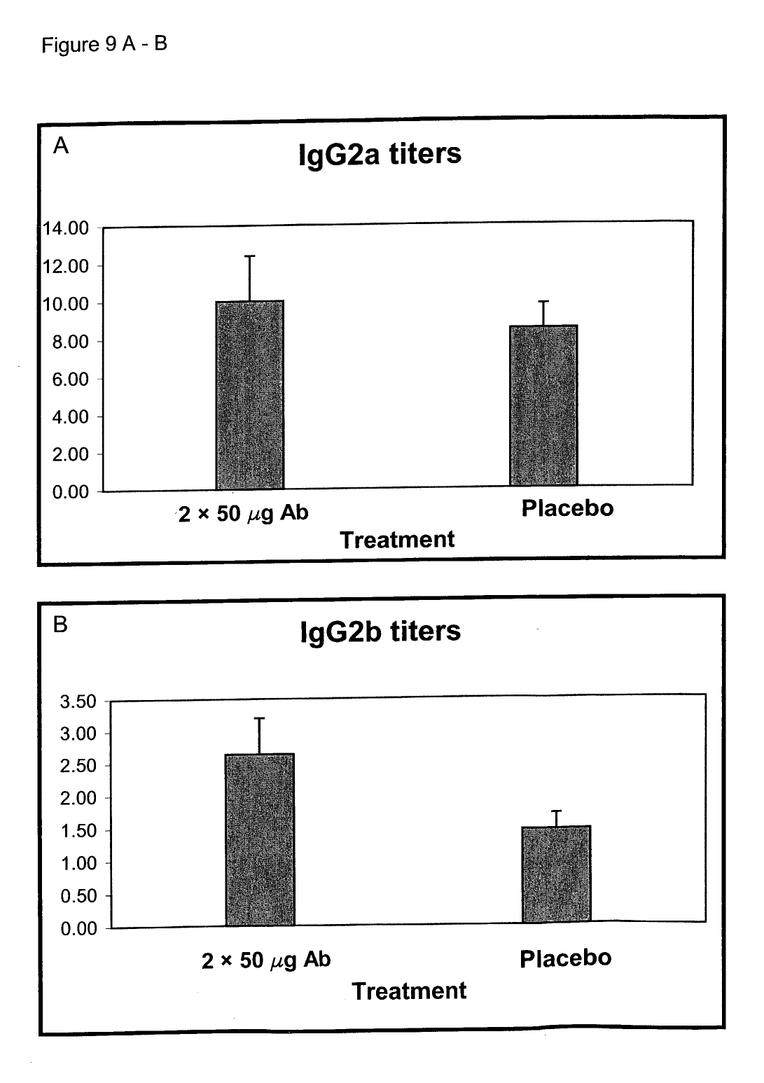

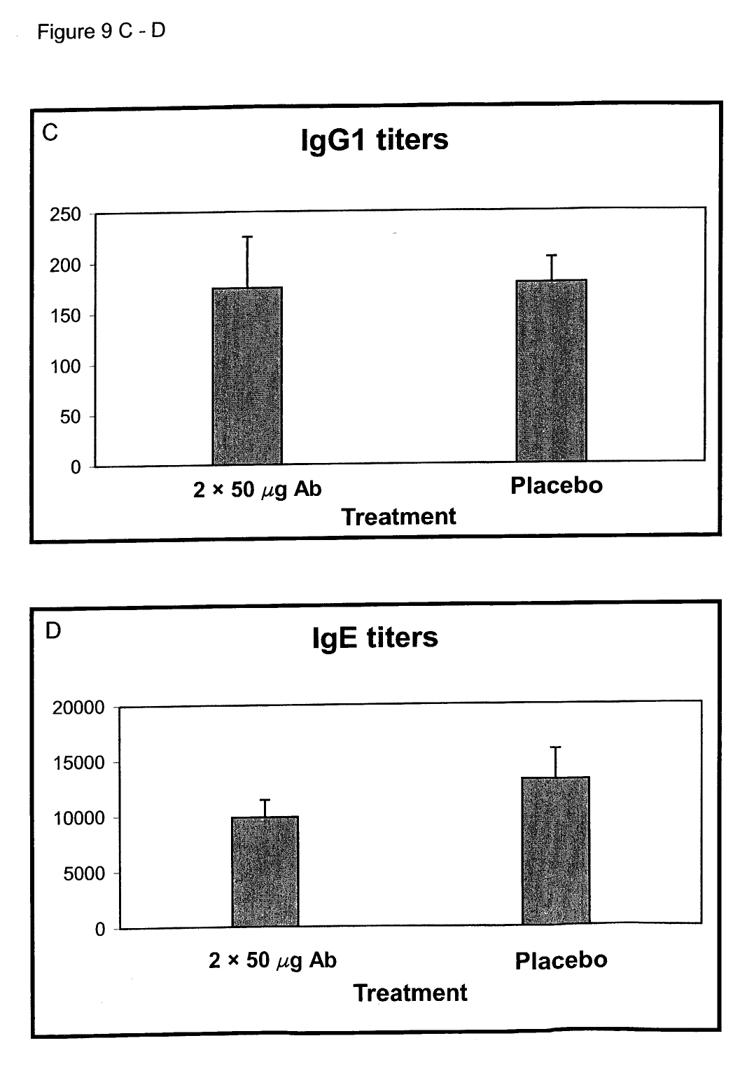

To verify whether the treatment with antibody affected the generation of new antibody-producing B cells and subsequently of new memory B cells, IgG2a treated mice were exposed to a second round of aerosol after a 2 week rest period. A marked increase of the Th1 -dependent IgG2a and IgG2b isotypes was observed in the treated mice (FIG. 9). The Th2-dependent isotypes remained at the same level (IgG1) or showed a slight decrease (IgE). Thus, although the mice did not receive an intermittent treatment with antibody, the memory IgE response (Th2 dependent) of the mice was reduced, whereas the memory IgG2 response (Th1 dependent) of the mice was enhanced. Accordingly, the treatment with anti-allergen IgG2 at the time of the first challenge not only reduced the airway inflammatory response to aeroallergen, but also selectively affected the formation of Th1 versus Th2-dependent memory B cells.

Example 6

### Persistence of the Reduced Airway Inflammatory Response to Aeroallergen During a Second Round of Aerosol Challenge

The previous observations that locally administered anti-allergen IgG2 protects against allergen-induced airway inflammation through an active instead of passive mechanism (see examples 3-5) implies that a modification of the nature of the anti-allergen immune response drives the airway eosinophilic inflammation. If this implication is true, a likely consequence would be that the immune response retains a memory of the altered nature and may cause a persistence of the therapeutical effect.

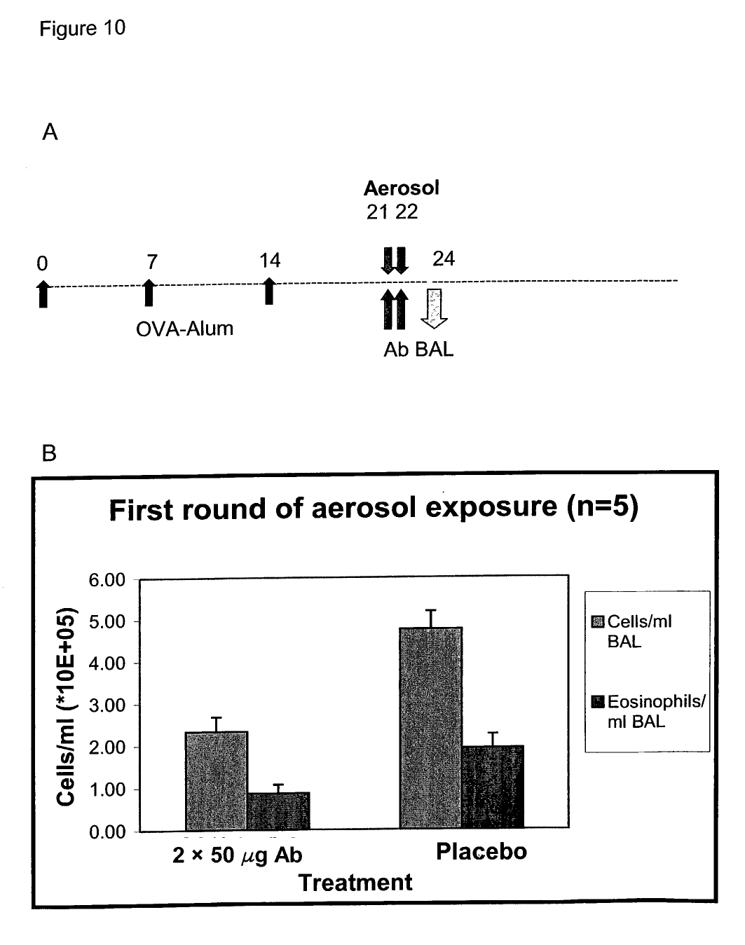

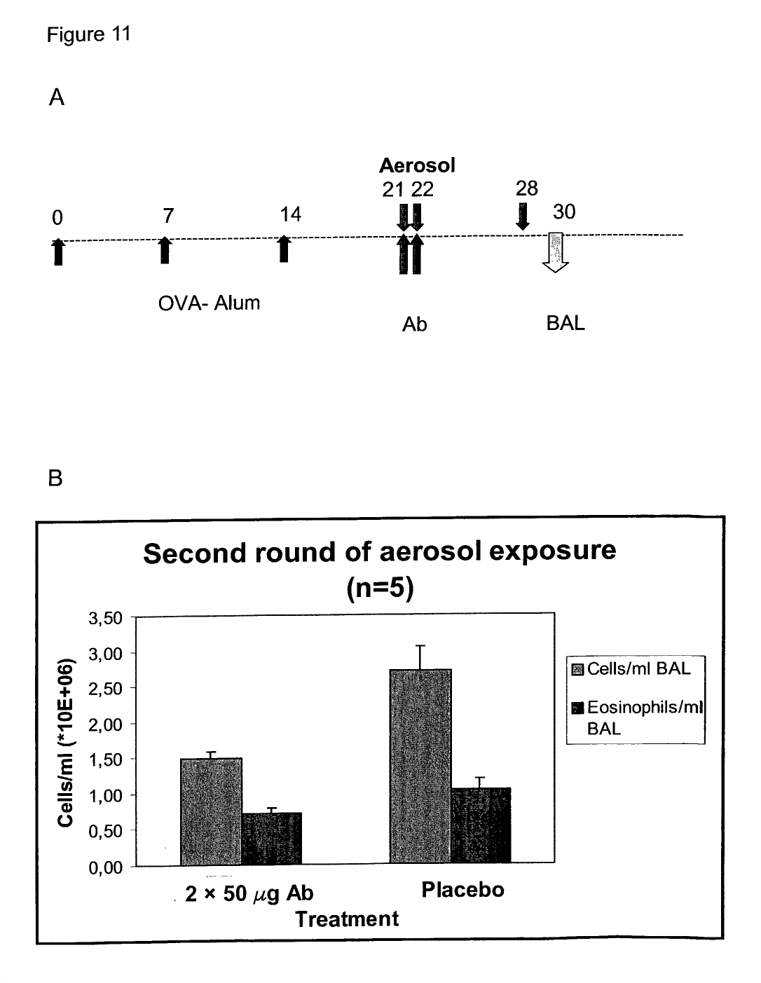

To verify this possibility, sensitized mice received a first challenge with OVA by exposure to aerosol during two consecutive days along with two separate administrations of 50 μg anti-OVA IgG2a given 2 h before each aerosol (FIG. 10). This treatment with antibody resulted in a pronounced reduction of bronchial alveolar cell infiltration and eosinophilia measured 48 h after the last OVA aerosol (FIG. 10). The persistence of the protective effect was verified by exposing the treated mice to a second round of aerosol challenge 6 days after the first round (FIG. 11). In this case, the mice did not receive an additional treatment with anti-OVA IgG2a which allowed analysis of the endurance of the protective effect during a new allergen exposure. As shown in FIG. 11, the mice retained a memory of the first treatment as apparent from the significantly lower airway inflammation and eosinophilia induced by the second round of allergen challenge. This result confirms the active nature of the treatment method and the capacity of the method to generate a sustained cure for asthma rather than a symptomatic treatment.

### Example 7

### Conversion of the Anti-allergen CD4⁺ T Cell Response from a Th2 Polarized Response to a Th1 and Th2 Mixed Response

The reduced airway inflammatory response, the persistent nature of the reduction and the increased formation of Th1-dependent memory B-cells, but not of Th2-dependent memory B-cells after intranasal administration of anti-allergen IgG2, indicate that an increased participation of Th1 cells represents the actual modification of the anti-allergen immune response that is responsible for the reduced asthmatic phenotype. To verify this possibility, the number of OVA-responsive Th1 and Th2 cells in the BAL was determined. BAL cells were stimulated in vitro with anti-CD3 antibody in the presence of anti-CD28 antibody (maximization of T cell costimulation) and the number of IFN-γ, IL-4 and IL-5-secreting CD4⁺ T cells were determined by cytoplasmic cytokine staining and 2-color flow cytometry (Table 2).

CD4⁺ T cells from the BAL of sensitized mice challenged with OVA and treated with placebo produced predominantly the Th2 cytokine IL-4, whereas a smaller fraction of the cells produced the Th1 cytokine IFN-γ. The prevailing Th2 nature of the bronchial alveolar CD4⁺ T cells is in agreement with the well-established Th2 nature of the airway inflammatory response. Treatment with anti-OVA IgG2a reversed the immune response to a prevailing Th1 response as apparent from the reduced number of IL-4-secreting Th2 cells, the doubling of IFN-γ-secreting Th1 cells and the resulting shift in the Th1/Th2 ratio from 0.5 to 1.9. From this result, it can be concluded that the anti-OVA IgG2a exerts its protective and sustained effect on allergen-induced airway eosinophilia by altering the Th1/Th2 ratio of the immune response and shifts the response pattern from a pathological Th2 response towards a benign Th1 response.

Intranasal administration, prior to aeroallergen exposure, of a compound that binds inhaled allergen and allows the inhaled allergen to be directed to antigen-presenting cells that induce and/or support Th1 cell responses and counteract Th2 responses, in casu IgG2 and macrophages (such as IFN-γactivated macrophages), respectively, has an inhibitory effect on aeroallergen-induced eosinophilic airway inflammation in sensitized mice by modifying the CD4⁺ T cell response from a predominant Th2 response to a predominant Th1 response.

TABLE 2
% Cytokine-positive CD4⁺ T cells from BAL.

| | |
|---|---|
| In vitro stimulation | In vivo treatment |

| Anti-CD3/anti-CD28 | Placebo | 2 × 50 μg Ab |
|---|---|---|
| IFN-γ | 5.10 | 11.18 |
| IL-4 | 9.39 | 5.79 |
| IL-5 | 2.35 | 2.17 |
| IFN-γ/IL-4 | 0.54 | 1.93 |

### Example 8

### Cross-protection to Unrelated Allergens

To verify whether the generation of a prevailing Th1 environment by local treatment with anti-allergen IgG2 promotes the generation of a prevailing Th1 response against unrelated aeroallergens, mice were simultaneously rendered sensitive to two inhaled antigens, OVA and human catalase (hCat). The occurrence of cross-protection was analyzed by intranasal administration of IgG2a antibodies against allergen, followed 2 h and 26 h later by intratracheal instillation of both antigens. Analysis of the BAL 2 days later reveals a clear reduction in airway inflammation as apparent from the reduced cell infiltration and degree of eosinophilia. This reduction was not observed in mice treated with the mismatched antibody which confirms the requirement for a high-affinity interaction between the administered IgG2a antibody and the allergen.

To verify the occurrence of cross-protection, the mice were exposed to aeroallergen 6 days after the last challenge. However, the aeroallergen was mismatched with respect to the specificity of the antibody instilled during the first round of allergen challenge. In this manner, mice treated with anti-hCat IgG2a and challenged with hCat and OVA were rechallenged with OVA without further treatment with antibody. Inversely, mice treated with anti-OVA IgG2a and challenged with hCat and OVA were rechallenged with hCat. In both instances, a reduction of the BAL cell infiltration and airway eosinophilia was observed despite the mismatch between the treating antibody given during the first challenge and the allergen instilled during the second challenge. These results demonstrate that an increase of Th1 reactivity against a single allergen exerts a bystander activity on the immune response against a second allergen and promotes the induction of a Th1 response against the second allergen. As a consequence, treatment concomitantly suppresses airway hyperreactivity to unrelated inhaled allergens through this bystander activity although the treatment specifically targeted a single allergen.

### REFERENCES

Azuma, I., K. Sugimora, T. Taniyama, M. Yawakawi, Y. Yamamura, S. Kusumoto, S. Okada, and T. Shiba. 1976. Adjuvant activity of mycobacterial fractions: Immunological properties of synthetic N-acetylmuramyl dipeptide and related compounds. Infect. Immun. 14:18-27.

Brewer, J. M., J. Richmond, and J. Alexander. 1994. The demonstration of an essential role for macrophages in the in vivo generation of IgG2a antibodies. Clin. Exp. Immunol. 97:164-171.

Corry, D. B., and F. Kheradmand. 1999. Induction and regulation of the IgE response. Nature 402-supp: B18.

de Jong, R., A. A. Janson, W. R. Faber, B. Naafs and T. H. Ottenhoff. 1997. IL-2 and IL-12 act in synergy to overcome antigen-specific T cell unresponsiveness in mycobacterial disease. J. Immunol. 159:786-793.

Donckier, V., D. Abramowicz, C. Bruyns, S. Florquin, M. L. Vanderhaeghen, Z. Amraoui, C. Dubois, P. Vandenabeele, and M. Goldman. 1994. IFN-gamma prevents Th2 cell-mediated pathology after neonatal injection of semiallogenic spleen cells in mice. J. Immunol. 153:2361-2368.

Gajewski, T. F., M. Pinnas, T. Wong, and F. W. Fitch. 1991. Murine Th1 and Th2 clones proliferate optimally in response to distinct antigen-presenting cell populations. J. Immunol. 146:1750-1758.

Galli, S. J. 2000. Allergy. Current Biology 10:R93.

Heinzel, F. P., M. D. Sadick, S. S. Mutha, and R. M. Locksley. 1991. Production of IFN-γ, IL-2, IL-4 and IL-10 by CD4⁺ lymphocytes in vivo during healing and progressive murine leishmaniasis. Proc. Natl. Acad. Sci. USA 88:7011-7015.

Hofstra, C. L., I. Van Arke, G. Hofman, M. Kool, F. P. Nijkamp, and J. M. Van Oosterhout. 1998. Prevention of Th2-like cell responses by co-administration of IL-12 and IL-18 is associated with inhibition of antigen-induced airway hyperresponsiveness, eosinophilia and serum IgE levels. J. Immunol. 161: 5054-5060.

Holt, P. G., C. Macaubas, P. A. Stumbles, and P. D. Sly. 1999. The role of allergy in the development of asthma. Nature 402-supp: B12.

Kalinkski, P., J. H. N. Schuitemaker, C. M. U. Hilkens, E. A. Wierenga, and M. L. Kapsenberg. 1999. Final maturation of dendritic cells is associated with impaired responsiveness to IFN-g and to bacterial IL-12 inducers: Decreased ability of mature dendritic cells to produce IL-12 during the interaction with Th cells. J. Immunol. 162:3231-3236.

Leonard, J. P., K. E. Waldburger, and S. J. Goldman. 1995. Prevention of experimental autoimmune encephalomyelitis by antibodies against interleukin 12. J. Exp. Med. 181:381-386.

Macatonia, S. E., N. A. Hosken, M. Litton, P. Vieira, C. S. Hsieh, J. A. Culpepper, M. Wysocka, G. Trinchieri, K. M. Murphy, and A. O'Garra. 1995. Dendritic cells produce IL-12 and direct the development of Th1 cells from naive CD4⁺ T-cells. J. Immunol. 154:5071-5079.

Merchant, M. A., Z. Zhu, J. Q. Yuan, A. Goddard, C. W. Adams, L. G. Presta and P. Carter. 1998. An efficient route to human bispecific IgG. Nature Biotechnology 16: 677-681.

Mosmann, T. R., and R. C. Coffman. 1989. Th1 and Th2 cells: different patterns of lymphokine secretion lead to different functional properties. Annu. Rev. Immunol. 7:145-173.

Murphy, J. W., B. A. Wu-Hsieh, L. M. Singer-Vermes, A. Ferrante, S. Moser, M. Ruso, S. A. Vaz, E. Burger, V. L. Calich, and I. C. Kowanko. 1994. Cytokines in the host response to mycotic agents. J. Med. Vet. Mycol. 32 suppl 1:203-210.

Nabors, G. S., L. C. C. Afonso, J. P. Farrell, and P. Scott. 1995. Switch form a type 2 to a type 1 T helper cell response and cure of established Leishmania major infection in mice is induced by combination therapy with interleukin 12 and pentostam. Proc. Natl. Acad. Sci. USA 92:3142-3146.

Racke, M. K., A. Bonomo, D. E. Scott, B. Cannella, A. Levine, C. S. Raine, E. M. Shevach, and M. Rocken. 1994. Cytokine-induced immune deviation as a therapy for inflammatory autoimmune disease. J. Exp. Med. 180:1961-1966.

Racke, M. K., D. Burnett, S. H. Pak, P. S. Albert, B. Cannella, C. S. Raine, D. E. McFarlin, and D. E. Scott. 1995. Retinoid treatment of experimental allergic encephalomyelitis. IL-4 production correlates with improved disease course. J. Immunol. 154:450-458.

Ronchese, F., B. Hausmann, and G. Le Gros. 1994. Interferon gamma and interleukin 4 producing T cells can be primed on dendritic cells in vivo and do not require the presence of B cells. Eur. J. Immunol 24:1148-1154.

Roussel, E., M. C. Gingras, E. A. Grimm, J. M. Bruner, and R. P. Moser. 1996. Predominance of a type 2 intratumoral immune response in fresh tumor-infiltrating lymphocytes from human gliomas. Clin. Exp. Immunol. 105:344-352.

Schwarze, J., G. Cieslewicz, A. Joetham, L. K. Sun, W. N. Sun, T. W. Chang, E. Hamelmann and E. W. Gelfand. 1998. Antigen-specific immunoglobin A prevents increased airway responsiveness and lung eosinophilia after airway challenge in sensitized mice. Am. J. Respir. Crit. Care Med. 158: 519-525.

Vogel, G. 1997. New clues to asthma therapies. Science 276:1643-1646.

Wills-Karp, M. 1999. Immunologic basis of antigen-induced airway hyperresponsiveness. Annu. Rev. Immunol. 17:255-281.

Yamamura, M., K. Uyemura, and R. J. Deans. 1991. Defining protective responses to pathogens: cytokine profiles in leprosy lesions. Science 254:277-279.

Yamauchi, N., S. Matunobo, K. Ito, and T. Miyamoto. 1983. Experimental allergic asthma in guinea pigs induced by the passive transfer of antibodies to sensitized cells. Jap. J. Allergology 32, 1084-1092.

Yang, X., S. Wang, Y. Fan, X. Han, and J. Yang. 2000. Mycobacterium bovis BCG infection inhibits established allergic reaction in murine asthma-like model. FASEB J. 14, A 1065, 100.2.

What is claimed is:

1. A method for inducing a CD4⁺ Th1 immune response in a subject, said method comprising:
administering a compound to the subject, wherein said compound is capable of binding an allergen and directing said allergen to an antigen-presenting cell; and
wherein said administering said compound induces and/or supports a Th1 response and counteracts a Th2 response.

2. A method for reducing aeroallergen-induced airway hyperreactivity in a subject, said method comprising:
administering a compound to the subject, wherein said compound is capable of binding an allergen and directing said allergen to an antigen-presenting cell; and
wherein said administering said compound induces and/or supports a Th1 response and counteracts a Th2 response.

3. The method according to claim 2, wherein the reduction of aeroallergen-induced airway hyperreactivity is persistent.

4. The method according to any one of claims 1-3, wherein said antigen-presenting cell is a macrophage.

5. The method according to any one of claims 1-4, wherein said compound is an IgG isotype antibody and not an IgG1 isotype antibody.

6. The method according to claim 5, wherein said compound is an IgG2 isotype antibody.

7. The method according to claim 5 or 6, wherein said antibody is an anti-allergen antibody.

8. The method according to any one of claims 1-7, wherein said compound is administered intranasally.

9. A pharmaceutical composition for treating a disease characterized by the natural CD4⁺ Th1/CD4⁺ Th2 balance being biased towards a Th2 response and/or which can be treated by shifting the balance towards a Th1 response, said pharmaceutical composition comprising: an IgG isotype antibody which is not an IgG1 isotype.

10. The pharmaceutical composition of claim 9, wherein said pharmaceutical composition is substantially free from other isotype antibodies.

12. The pharmaceutical composition of claim 9, wherein said disease is allergic asthma.

13. The pharmaceutical composition of claim 9, wherein said disease is allergic rhinitis.

14. The pharmaceutical composition of claim 9, wherein said disease is airway hyperreactivity and/or eosinophilic airway inflammation.

15. The pharmaceutical composition of any one of claims 9-14, wherein said at least one IgG antibody is an anti-allergen antibody.

16. The pharmaceutical composition of claim 9, wherein the IgG isotype antibody is directed against antigenic structures of causative agents of the disease.

17. A pharmaceutical composition for treating a disease characterized by the natural CD4⁺ Th1/CD4⁺ Th2 balance being biased towards a Th2 response and/or which can be treated by shifting the balance towards a Th1 response, said pharmaceutical composition comprising:
at least one IgG isotype antibody substantially free from other isotype antibodies; and
wherein said at least one IgG isotype is not an IgG1 isotype antibody.

18. The pharmaceutical composition of claim 17, wherein said at least one IgG antibody is an anti-allergen antibody.

19. The pharmaceutical composition of claim 17, wherein the pharmaceutical composition is configured for intranasal administration.
